(19) United States
(12) Patent Application Publication
(10) Pub. No.: US 2009/0214620 A1
(43) Pub. Date: Aug. 27, 2009

(54) AGENT FOR USE IN THE CASE OF DISORDERS OF BLOOD SUGAR METABOLISM, INCLUDING DIABETES

(75) Inventors: Daniel Henry Wyrobnik, Frankfurt am Main (DE); Isaac Harry Wyrobnik, Frankfurt am Main (DE)

Correspondence Address: HESLIN ROTHENBERG FARLEY & MESITI PC, 5 COLUMBIA CIRCLE, ALBANY, NY, 12203, US

(73) Assignee: PRO NATURA GESELLSCHAFT FUER GESUNDE ERNAEHRUNG MB, Frankfurt am Main (DE)

(21) Appl. No.: 12/094,684

(22) PCT Filed: Nov. 23, 2006
(86) PCT No.: PCT/EP06/11232

§ 371 Date: Oct. 21, 2008

(30) Foreign Application Priority Data
Nov. 23, 2005 (DE) 10 2005 056 103.9
Dec. 20, 2005 (DE) 10 2005 061 329.2
Jan. 5, 2006 (DE) 10 2006 001 017.5
Mar. 27, 2006 (DE) 10 2006 014 424.4

(51) Int. Cl. A61K 47/00; A61K 38/44

(52) U.S. Cl. 424/439; 424/94.4

(57) ABSTRACT

An agent for use in the case of disorders of blood sugar metabolism, including diabetes, is described, which reduces the glucose content of food and other substances with the help of 5-D-fructose dehydrogenase and glucose isomerase.

The present invention relates to the use of an agent in the case of diabetes, which reduces the content of glucose in food and other substances consumed. In the context of this application, the term “diabetes” means all forms of disorders of blood sugar metabolism, also of the mild type, including all forms of diabetes, such as diabetes type I, including LADA diabetes (latent autoimmune diabetes in adults), diabetes type II, pregnancy-induced diabetes and impaired glucose tolerance. Accordingly, the invention is also suitable for persons who only have minor disorders of blood sugar metabolism and, therefore, are not (yet) referred to as diabetics in the medical sense.

According the invention the term “agent” includes a pharmaceutical composition, a medical device, a foodstuff and a special foodstuff.

According to the present invention, the terms food and foodstuff are used as synonyms. They mean to also include feed in the sense of animal feed. In the context of this application special foodstuffs are foodstuffs for particular nutritional uses, foods for special medical purposes, medical foods, food supplements, dietary supplements, dietetic food supplements, health foods, nutraceuticals and food additives. In the context of this application the term foodstuff means to include special foodstuffs as used herein, where applicable.

In the context of this application the term “glucose containing” refers to all substances and foodstuffs that either contain glucose in pure form or from which glucose can be released in the digestive tract. The glucose content of substances and foodstuffs refers to all the glucose in a glucose containing food or substance in whatever form (e.g. also as part of sucrose) it is contained in such a food or substance. From sucrose, glucose and fructose are released in equal parts by enzymatic cleavage in the intestines. In the context of this application the term “fructose containing” refers to all substances and foodstuffs that either contain fructose in pure form or from which fructose can be released in the digestive tract. The fructose content of substances and foodstuffs refers to all the fructose in a fructose containing food or substance in whatever form (e.g. also as part of sucrose) it is contained in such a food or substance.

Diabetes is an extremely wide-spread problem and the incidence of diabetes has constantly increased in Western Europe and North America. The two most important forms are diabetes type I (approx. 5 to 10%) and diabetes type II (approx. 90%). In the Federal Republic of Germany, for example, more than 9.9 million people suffer from diabetes of which approx. 3.9 million people suffer from the so-called impaired glucose tolerance. The consequences of diabetes are an increase in the level of blood glucose, glucosuria and later sequelae of the increased level of blood glucose in different organ systems, disorders of lipid metabolism, etc. Depending on whether it is a question of the various damage caused by average blood sugar values being too high over many years, such as to the eyes, kidneys and nerves, or damage caused by short-term blood sugar peaks, such as premature damage to the walls of the large arterial vessels, one refers to micro- or macro-angiopathic sequelae. The list of possible complications and late damage caused by diabetes is long and they are described in detail in the specialist literature.

Until now, therapies for disorders of blood sugar metabolism have basically been limited to increasing the content of insulin in the blood (by administering insulin or by enhancing the excretion of insulin) and enhancing the efficiency of insulin. An alternative approach is to delay the uptake of carbohydrates (agent for delaying absorption), but this leads to abdominal pain due to the bacterial degradation of carbohydrates that accumulate in the lower sections of the intestines. Despite all methods found to date for treating diabetes, there is still a need for agents that prevent or reduce the increase in the level of blood sugar as a result of the intake of glucose containing foods and substances, without placing a burden on the body.

An agent that would reduce the absorbed amount of glucose would thus satisfy an extremely wide-spread and pressing need which has existed for decades, since sufferers have considerable difficulties with “individual adjustment”, i.e. with the exact adjustment of the dose of insulin in relation to the intake of carbohydrates. These difficulties remain, despite the variety of available medicaments and glucose free dietetic foodstuffs especially for diabetics, who are already very limited in the choice of foodstuffs they can eat. Such an agent would overcome the prejudice widely held in the specialist world and among those suffering from diabetes that a low-carbohydrate diet has to be maintained and that a considerable change in nutritional habits is necessary, and would also mean a significant improvement and dramatic development in the therapeutic and nutritional options in diabetes. Such an agent would also put an end to the as yet fruitless efforts of the specialist world to find an agent to treat diabetes which can be administered broadly, easily, and long-term, without causing side effects. This would apply all the more to an agent which, in addition, has no negative effects on health.

Thus, it is an object of the present invention to provide an agent that significantly reduces the usable content of glucose in food, in particular for facilitating the intake of foodstuffs that normally contain glucose also in the case of diabetes, without resulting in negative effects on health. Further, it is an object of the invention to make it possible for diabetics to eat foodstuffs which until now were not allowed to them due to their glucose content or the eating of which was associated with negative effects for the sufferers' health. Moreover, the objective is to provide an agent that can reduce or prevent the associated negative effects on health after the intake of glucose in diabetes. Another object of the invention is to provide an agent that can reduce or prevent an increase in the level of blood glucose in diabetes after the intake of glucose containing food.

These objects are solved by the subject matter as described in claims 1 to 230. Therefore, the subject matter of the invention is an agent that can solve all of the problems described above. The invention relates to the use of an agent for the curative or prophylactic treatment of diabetes, for diagnosis of diabetes, for reducing the bioavailability of glucose and/or fructose in the human or animal body, for reducing the glucose content and/or the fructose content in a foodstuff, and for preventing or at least reducing an increase of the level of blood glucose after the intake of glucose containing food.

The agent contains 5-D-fructose dehydrogenase (syn. fructose 5-dehydrogenase) and glucose isomerase. The enzyme 5-D-fructose dehydrogenase effects the conversion of fructose into 5-keto-D-fructose Glucose isomerase has the property of converting glucose into fructose and vice versa with an equilibrium concentration of approximately 50% glucose and 50% fructose. In the context of this application, a 5-D-fructose dehydrogenase is an enzyme that can catalyze the dehydrogenation of fructose to 5-keto-D-fructose. A glucose isomerase, in the context of this application, is an enzyme that is able to transform glucose into fructose. This conversion can also be brought about, for example, by a xylose isomerase. Thus, such a xylose isomerase is, in the sense of this invention, also a glucose isomerase. A possible method for the production of a xylose isomerase is, for example, described in Yamanaka, Biochimica et Biophysika Acta, Volume 151 (3), 1968, 670-680, “Purification, Crystallization and Properties of the D-Xylose Isomerase from Lactobacillus brevis” and in Yamanaka, Methods in Enzymology, Volume 41, 1971, 466-471, “D-Xylose Isomerase from Lactobacillus brevis”.

The effect of the enzyme combination according to the present invention will be explained using starch as an example. Since in particular glucose is released from starch as a monosaccharide during digestion, this is transformed by the glucose isomerase into fructose, which is then converted by the 5-D-fructose dehydrogenase into 5-keto-D-fructose, which cannot be metabolized by the body. Thus, the 5-D-fructose dehydrogenase prevents the establishment of the above-mentioned equilibrium. Therefore, said glucose isomerase will convert glucose into fructose, which itself will be dehydrogenated into 5-keto-D-fructose by the 5-D-fructose dehydrogenase, until no further glucose is present in the food or food pulp.

Also in the case of sucrose, a reduction of glucose can be achieved with this combination agent. Fructose which is released from sucrose during digestion is converted into 5-keto-D-fructose by the 5-D-fructose dehydrogenase, as described above. The Glucose isomerase then tries to balance out the resulting “disequilibrium” by converting glucose into fructose. As in the case of starch, this conversion process continues until no further glucose is present in the food pulp. It is possible that part of the glucose has already been absorbed by then. However, the total amount of glucose that will be absorbed from a sugar containing meal will be significantly reduced with the help of the invention disclosed herein. Depending on the level of enzyme activity per dose unit and the amount of the glucose content of the respective meal, it is possible to influence the amount of glucose absorbed by the body. If desired, according to the present invention, it is also possible to achieve a complete or virtually complete elimination of glucose.

Thus, the invention is based on the fact that the glucose contained in the consumed carbohydrates, such as sucrose, or released from them in the intestines, is no longer available for the undesired absorption from the intestines and release into the bloodstream. This is achieved by consuming a mixture of the two enzymes glucose isomerase and 5-D-fructose dehydrogenase before, shortly before, with, shortly after or after consumption of glucose-containing-foods, by conversion of glucose to fructose and its subsequent dehydrogenation to 5-keto-D-fructose. The enzymes transform glucose into fructose and the fructose into 5-keto-D-fructose, until no further glucose is present. The dosage of the enzymes added may be selected in such a way that, also in the case of an intake of larger amounts of glucose, the reaction can take place at an appropriate rate.

Furthermore, the enzyme combination according to the present invention also has the effect of reducing the intake of calories, which is desired in the case of diabetes, since the enzyme combination, as described above, converts carbohydrates contained in the food into 5-keto-D-fructose, which is significantly less caloric than fructose and glucose. In particular, this is desired in the case of diabetes type II, since diabetics often suffer from obesity, hypertension and disorders of lipid metabolism. In addition, the enzyme combination according to the present invention has the effect that it can convert fructose originating from fructose containing substances and foodstuffs into 5-keto-D-fructose, which is desired in the case of diabetes. Although fructose is used in large amounts as a sweetener in food for diabetics, and fructose is generally regarded as being well-tolerated and harmless for diabetics, there is an ongoing debate among specialists that the intake of fructose is also undesirable and contraindicated in diabetes and should be limited, in particular due to the utilizable calorie content of fructose. In contrast to glucose, fructose is metabolized independently of insulin. Since insulin influences the occurrence of the sensation of satiation indirectly, fructose does not eliminate the appetite, obesity may easily occur as a result of the extensive use of fructose as a sweetener. Free fructose in large amounts may also favour hypertension. It also influences the lipid profile (blood lipids) in an unfavourable way, since in larger amounts it promotes the synthesis of lipids and thus increases the postprandial serum triglycerides. Therefore, especially larger amounts of fructose should not be consumed by diabetics.

Patients with metabolic syndrome are advised in particular not to consume beverages that are sweetened with HFCS (high fructose corn syrup) or sucrose.

Therefore, a subject matter of the invention is an agent for use in the case of diabetes which contains a glucose isomerase and a 5-D-fructose dehydrogenase.

Further, a subject matter of the invention is an agent that reduces the bioavailability of glucose in the human or animal body with the help of a glucose isomerase in combination with a 5-D-fructose dehydrogenase.

Also, a subject matter of the invention is an agent for reducing the utilizable content of glucose of food, which contains a glucose isomerase in combination with a 5-D-fructose dehydrogenase.

Also, a subject matter of the invention is an agent for preventing or reducing an increase of the level of blood glucose in the case of diabetes after the intake of glucose containing food.

A further subject matter of the invention is the use of a glucose isomerase in combination with a 5-D-fructose dehydrogenase in the case of diabetes and in the case of health problems and diseases associated with diabetes.

According to the present invention, a 5-D-fructose dehydrogenase in combination with glucose isomerase can also be used for reducing the utilizable content of glucose in a foodstuff.

In a particularly easy way, the invention facilitates the transformation of glucose in a foodstuff into a form that does not result in an increase in the level of blood glucose. Thus, the invention enables diabetics to consume foodstuffs that they have had to avoid up till now, due to their glucose content.

According to the present invention, 5-D-fructose dehydrogenase in combination with glucose isomerase is further mentioned for use in medicine, for example, as a pharmaceutical composition. Accordingly, a subject matter of the invention is also a product that consists of 5-D-fructose dehydrogenase in combination with glucose isomerase or contains 5-D-fructose dehydrogenase in combination with glucose isomerase, beside one or more other active ingredients, for use in a medical method, especially in a method for the therapeutic treatment of the human or animal body. In the context of this application, a pharmaceutical composition is a product, especially a substance or a substance mixture, for use in a method for surgical or therapeutic treatment of the human or animal body and in diagnostic methods that are performed on the human or animal body. Thus, in the sense of the invention, pharmaceutical compositions are also products, in particular substances or substance mixtures, that are meant or suitable for curing, alleviating, preventing or determining diabetes.

The term “treating” when used in connection with the foregoing disorders includes amelioration, prevention or relief from the symptoms and/or effects associated with these disorders and includes the prophylactic administration of an enzyme or a mixture thereof to diminish the likelihood or seriousness of the conditions.

According to a further aspect, according to the present invention, a foodstuff is provided that contains glucose isomerase in combination with 5-D-fructose dehydrogenase. Further, according to the present invention, a foodstuff is provided that contains 5-D-fructose dehydrogenase in combination with glucose isomerase in an amount which is effective for converting fructose into 5-keto-D-fructose and glucose isomerase in an amount which is effective for transforming glucose into fructose. Such a foodstuff may be produced advantageously using a method for treating a foodstuff in which the foodstuff is placed in contact with a 5-D-fructose dehydrogenase in combination with glucose isomerase under such conditions under which the 5-D-fructose dehydrogenase can dehydrogenate fructose to 5-keto-D-fructose and the glucose isomerase can convert glucose into fructose. In contrast to otherwise untreated foodstuffs, such a foodstuff has a reduced content of glucose and therefore, for the first time, is suitable to be eaten by diabetics. Particularly advantageously, a foodstuff may be prepared by a method in which a glucose isomerase in combination with a 5-D-fructose dehydrogenase is added to the foodstuff in a manner in which the action of the two enzymes does not start until after the foodstuff has been consumed. Such a foodstuff that contains 5-D-fructose dehydrogenase and glucose isomerase has the same taste as an untreated food-stuff and is, for the first time, suitable to be eaten by diabetics, due to the reduced content of glucose which is established after consumption.

According to a further aspect, according to the present invention, 5-D-fructose dehydrogenase in combination with glucose isomerase is provided as a medical device. Accordingly, the subject matter of the invention is also a medical device that consists of 5-D-fructose dehydrogenase in combination with glucose isomerase or contains 5-D-fructose dehydrogenase in combination with glucose isomerase, beside one or more other active ingredients.

In the following, the invention will be described further in its various aspects.

5-D-fructose dehydrogenase is a compound that has been known for nearly 40 years, but has only been used for analytical purposes to date. Glucose isomerase is a compound that has been known for more than 40 years and has only been used for starch saccharification to date. In the industry, it is used for the conversion of glucose into fructose as well as for the conversion of fructose into glucose.

Until now, 5-D-fructose dehydrogenase has not been used in combination with glucose isomerase in the case of diabetes of humans or animals.

The agent according to the present invention can be taken orally prior to meals, immediately before meals, with meals or immediately after meals, so that it can exert its converting effect on glucose and dehydrogenating effect on fructose in the food pulp. The agent according to the present invention may contain the enzymes without further additives. However, it is preferable that the agent according to the present invention further contains additives that are pharmaceutically acceptable and/or acceptable for foodstuffs, such as for example extenders, binders, stabilizers, preservatives, flavourings, etc. Such additives are commonly used and well known for the production of pharmaceutical compositions, medical devices, food-stuffs, and special foodstuffs and the person skilled in the art knows which additives in which amounts are suitable for certain presentation forms. The agents according to the present invention may for example contain as additives dicalcium phosphate, lactose, modified starch, microcrystalline cellulose, maltodextrin and/or fibersol.

The agent according to the present invention can also be added to a foodstuff before its consumption. It can already be added to the foodstuff during production, with the aim that it exhibits its effect only after eating the foodstuff. This could also be achieved by microencapsulation, for example. With this, the utilizable glucose content of the foodstuff would be reduced without negatively affecting its taste. Therefore, preparations containing 5-D-fructose dehydrogenase and glucose isomerase are useful, which release this enzyme only in the digestive tract of a human or animal or let it become effective in another way, especially in the stomach or small intestine. Therefore, the invention can be used, for example, in the production of desserts, fruit preparations (e.g. apple sauce), jam, honey, chocolate and chocolate products, bakery products (e.g. biscuits and cakes), breads, pastas, vegetable dishes, potato dishes, ice cream, cereals, dairy products (e.g. fruit yogurt and pudding), fructose- and/or glucose-containing beverages, fructose- and/or glucose-containing sauces (e.g. tomato ketchup) and fructose- and/or glucose-containing sweeteners. For dishes that are boiled or baked, the agent according to the present invention could e.g. be mixed into or sprinkled onto them after cooling.

The agent according to the present invention can also be added to a foodstuff, to exert its effect after eating on the glucose originating from another foodstuff. An example of this would be the addition of the agent according to the present invention to a spread so that the reduction of the glucose that is contained in the bread and that can be used by the body occurs after the intake of the bread, without impairing its taste. Further examples would be mixed spices and mayonnaise for use with french fries.

The agent according to the present invention may also be used in immobilized form. This is especially useful for the treatment of liquid foodstuffs. For example, the enzymes can be embedded in a matrix which is permeable for glucose. If a glucose containing liquid foodstuff is allowed to flow along the enzyme containing matrix, then glucose is extracted from the foodstuff by the action of the enzymes and converted to 5-keto-D-fructose.

A subject matter of the present invention are also agents that, in addition to other active ingredients, also contain glucose isomerase in combination with 5-D-fructose dehydrogenase .

The agent may be formulated in any form which is suitable for the intended route of administration. A preferred route of administration is oral administration.

For oral administration, the agent may be formulated for example in the form of capsules (coated or non-coated) containing powder, coated or non-coated pellets, granules or micro-/mini-tablets or in the form of tablets (coated or non-coated) pressed from powder, coated or non-coated pellets, dragées or micro-/mini-tablets. The agent may also be formulated for example in the form of gel caps or in liquid form as solution, drops, suspension or gel. The agent may also be formulated e.g. as dried or moist oral supplement. The formulation of the agent according to the present invention as powder is particularly suitable for admixing with foodstuff. The powder may be sprinkled onto a meal or mixed into a pulp or beverage. It is particularly beneficial, if the agent offered as bulk powder is packaged in single dosage amounts, such as in single bags or capsules, or if it is provided in a dosing dispenser.

For oral administration, the 5-D-fructose dehydrogenase in combination with glucose isomerase may be used with acceptable excipients and/or carriers.

The total amount of the carrier and/or excipient of an agent containing 5-D-fructose dehydrogenase and glucose isomerase is preferably between 5 and 99.9 % by weight, more preferably between 10 and 80% by weight and even more preferably between 25 and 60% by weight of the composition.

Suitable excipients and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethyl cellulose, corn starch, modified starch, fibersol, gelatine, hydroxypropylmethyl cellulose and the like (including mixtures thereof).

Preferable carriers include calcium carbonate, magnesium stearate, maltodextrin, dicalcium phosphate, modified starch, microcrystalline cellulose, fibersol, gelatine, hydroxypropylmethyl cellulose and mixtures thereof.

The various ingredients and the excipient and/or carrier may be mixed and formed into the desired form using common methods well known to the skilled person. The administration form according to the present invention which is suited for the oral route, such as e.g. tablet or capsule, may be coated with a coating which is resistant against low pH values (approximately pH 1 to 2.5) and which dissolves at a pH value of approximately 3.0 to 8.0, preferably at a pH value of 3.0 to 6.5 and particularly preferable at a pH value of 4.0 to 6.0. An optionally used coating should be in accordance with the pH optimum of the enzyme used and its stability at pH values to which the formulation will be exposed. Also a coating may be used which is not resistant to low pH values but which delays the release of the enzyme at low pH values. It is also possible to prepare the agent according to the present invention as coated (see above) pellets, granules or micro-/mini-tablets which can be filled into coated or non-coated capsules or which can be pressed into coated or non-coated tablets. Suitable coatings are, for example, cellulose acetate phthalate, cellulose derivates, shellac, polyvinylpyrrolidone derivates, acrylic acid, polyacrylic acid derivates and polymethyl methacrylate (PMMA), such as e.g. Eudragit® (from Röhm GmbH, Darmstadt, Germany), in particular Eudragit® L30D-55. The coating Eudragit® L30D-55 is dissolved, for example, at a pH value of 5.5 and higher. If it is desired to release the enzyme already at a lower pH value, this may be achieved e.g. by the addition of sodium hydroxide solution to the coating agent Eudragit® L30D-55, because in this case carboxyl groups of the methacrylate would be neutralised. Therefore, this coating will be dissolved, for example, already at a pH value of 4.0 provided that 5% of the carboxyl groups are neutralised. The addition of about 100 g of 4% sodium hydroxide solution to 1 kg of Eudragit® L30D-55 would result in a neutralisation of about 6% of the carboxyl groups. Further details about formulation methods and administration methods can be found in the 21st edition of “Remington: The Science & Practice of Pharmacy”, published 2005 by Lippincott, Williams & Wilkins, Baltimore, USA, in the Encyclopedia of Pharmaceutical Technology (Editor James Swarbrick) and in Prof. Bauer “Lehrbuch der Pharmazeutischen Technologie”, 18th edition, published 2006 by Wissenschaftliche Verlagsgesellschaft (ISBN 3804-72222-9). The contents of these documents are incorporated herein by reference.

Other suitable acceptable carriers or adjuvants for use in the present invention include, but are not restricted to water, mineral oil, ethylene glycol, propylene glycol, lanolin, glyceryl stearate, sorbitan stearate, isopropyl myristate, isopropyl palmitate, acetone, glycerine, phosphatidylcholine, sodium cholate or ethanol.

The compositions for use in the present invention may also comprise at least one co-emulsifying agent which includes but is not limited to oxyethylenated sorbitan monostearate, fatty alcohols, such as stearyl alcohol or cetyl alcohol, or esters of fatty acids and polyols, such as glyceryl stearate.

The agents according to the present invention may be provided in a stabilized form. Generally, stabilization methods and procedures which may be used according to the present invention include any and all methods for the stabilization of chemical or biological material which are known in the art, comprising e.g. the addition of chemical agents, methods which are based on temperature modulation, methods which are based on irradiation or combinations thereof. Chemical agents that may be used according to the present invention include, among others, preservatives, acids, bases, salts, antioxidants, viscosity enhancers, emulsifying agents, gelatinizers, and mixtures thereof.

Usually, the industrial production of enzymes is performed in a technical fermentation way using suitable microorganisms (bacteria, moulds, fungi). Usually the strains are recovered from natural ecosystems according to a special screening protocol, isolated as pure cultures as well as improved in their properties with respect to the enzyme spectrum and biosynthesis performance (volume/time yield). Enzyme production may also be carried out by methods developed in the future.

5-D-fructose dehydrogenase is commercially available (e.g. Sigma-Aldrich or Toyobo Enzymes, Japan) and is usually prepared in a microbiological way with the help of the microorganism gluconobacter industrius. Glucose isomerase is also commercially available (e.g. Sigma-Aldrich or Novozymes A/S, Denmark) and usually prepared in a microbiological way with the help of the microorganism streptomyces murinus. However, the invention is not limited to the enzymes that are commercially available at the moment, but generally relates to enzymes that can catalyze the conversion of fructose—specifically or non-specifically—to 5-keto-D-fructose, and of glucose—specifically or non-specifically—to fructose. A person skilled in the art can prepare suitable further enzymes by conventional methods, for example by mutagenesis of the gene encoding 5-D-fructose dehydrogenase which is present in gluconobacter industrius or by mutagenesis of the gene encoding glucose isomerase in streptomyces murinus. The enzymes may also be prepared with the help of other microorganisms, such as fungi, in sufficient amounts and the required purities, also by the use of the genetic engineering methods which are presently known or may be developed in the future. For example, if it is desired to produce the enzymes with other microorganisms, then the genetic information of a microorganism which has been found initially by extensive screening and which has been proven to be a suitable source of the enzyme with the desired properties can be transferred to a microorganism which is normally used for the production of enzymes. Also the modification of the enzymes and the production of the enzymes by means of methods which are presently known or may be developed in the future in the area of industrial enzyme development and enzyme production, such as genetic engineering, is possible. The use and the manner of performing all these methods for developing and producing the enzyme(s) with the desired purities and activities and with the desired properties, in particular with respect to the stability of the enzyme(s) at various pH values, regarding the optimum of the pH value, the stability at various temperatures and temperature optimum, are well known to a person skilled in the art. The explanations in chapter 2 (page 82 to page 130) of the textbook “Lebensmittel-Biotechnologie und Ernährung” of Heinz Ruttloff, Jürgen Proll and Andreas Leuchtenberger, published by Springer Verlag 1997 (ISBN 3-540-61135-5) describe these methods in detail. These methods are also described in “Advances in Fungal Biotechnology for Industry, Agriculture, and Medicine” by Jan S. Tkacz, Lene Langeand (published in 2004, ISBN 0-306-47866-8), in “Enzymes in Industry: Production and Applications” by Wolfgang Aehle (Editor), published in 2004, ISBN 3527295925 and in “Microbial Enzymes and Biotransformations” by Jose-Luis Barredo (Humana Press 2005, ISBN 1588292533). These documents are herewith incorporated into the patent application by reference. All this also applies to the enzymes mentioned below that can optionally be added to the agent according to the present invention.

The activity of 5-D-fructose dehydrogenase is defined in units (assay available e.g. from Sigma-Aldrich), whereby one unit is the amount of 5-D-fructose dehydrogenase that converts one micromole of D-fructose to 5-keto-D-fructose per minute at pH 4.5 and 37° C. Generally, the activity of 5-D-fructose dehydrogenase per dose unit should be between 10 and 5 million units, preferably between 25 and 2.5 million units and particularly preferably between 50 and 1 million units.

The activity of glucose isomerase should generally be between 0.01 and 100,000 GIU, preferably between 0.05 and 10,000 GIU and particularly preferably between 0.1 and 1,000 GIU per dose unit. One unit of this enzyme is defined as a glucose isomerase unit (GIU). One GIU converts 1 g of glucose into fructose at a pH value of 6.0 and at a temperature of 37° C. from a solution of initially 10% (percent by weight, i.e. 10 g of glucose +90 g of water) in 5 minutes.

The wide range of the above mentioned dosages may be explained by the fact that the agent according to the present invention can be applied in completely different types of diabetes in the whole range of different severities. Furthermore, the different dosages also result from the fact that strongly varying amounts of glucose are supplied, depending on the food in question.

The agent according to the present invention may comprise one or more additional enzymes, such as invertase (syn. beta-fructofuranosidase or beta-fructosidase), lactase (syn. beta-galactosidase), maltase (syn. alpha-glucosidase), alpha-amylase, beta-amylase, glucoamylase, pullulanase, isoamylase, amyloglucosidase, cyclomaltodextrin glucantransferase (CGTase). These enzymes have the property of releasing fructose and/or glucose from fructose and/or glucose containing substances and foodstuffs—alone or in combination with one or more of these enzymes—, whereby the enzymes pullulanase and isoamylase also increase the efficiency of glucoamylase and beta-amylase. All these enzymes are commercially available (e.g. BioCat Inc., Troy, USA or Novozymes A/S, Denmark or Amano Enzymes Inc., Japan or Sigma-Aldrich) and, up to now, have never been used in combination with 5-D-fructose dehydrogenase and glucose isomerase in the case of diabetes. Examples for agents according to the present invention include:

5-D-fructose dehydrogenase in combination with invertase, or 5-D-fructose dehydrogenase in combination with glucose isomerase and invertase, or 5-D-fructose dehydrogenase in combination with glucose isomerase and lactase and invertase, still further 5-D-fructose dehydrogenase in combination with glucose isomerase, Invertase, alpha amylase, beta amylase, glucoamylase, maltase, isoamylase and pullulanase (combination of 9 enzymes), or 5-D-fructose dehydrogenase in combination with glucose isomerase, alpha amylase, beta amylase, glucoamylase, maltase, isoamylase and pullulanase as well as invertase and lactase (combination of 10 enzymes).

For example, said invertase can release glucose from e.g. sucrose and lactase can release glucose from lactose. Beta-amylase breaks down e.g. 1,4-alpha-bonds in starch, starting at the non-reducing end of the polysaccharide chain with cleaving of maltose, and glucose is released by the action of maltase on maltose. By the addition of one or more of these enzymes to the agent according to the present invention, the endogenic release of glucose from glucose containing substances or foodstuffs, in particular from sucrose and starch, may also be promoted and accelerated, so that the conversion of glucose into fructose effected by the glucose isomerase and the conversion of fructose into 5-keto-D-fructose which is catalyzed by the 5-D-fructose dehydrogenase may occur earlier. Therefore, the addition of one or more of these enzymes to the agent according to the present invention may have the benefit of reducing the required amount of 5-D-fructose dehydrogenase and glucose isomerase.

The activity of invertase is measured in Sumner units (SU, assay available e.g. from Bio-Cat Inc., Troy, Va., USA). An SU is defined as the amount of the enzyme which converts 1 mg of sucrose into glucose and fructose under standard test conditions within 5 minutes at 20° C. and a pH value of 4.5. If the agent according to the present invention also contains invertase, the activity of the invertase per dose unit should be between 50 and 250,000 SU, preferably between 100 and 150.000 SU and particularly preferably between 150 and 100,000 SU per dose unit.

The activity of lactase is given in Food Chemical Codex (FCC) units (assay is published in the Food Chemical Codex, fifth edition, and also available e.g. from Bio Cat Inc. Troy, Va. or Amano Enzymes, Japan or from Sigma Aldrich). If the agent according to the present invention also contains lactase, the activity of the lactase per dose unit should be between 50 and 200,000 FCC units, preferably between 100 and 100,000 FFC units and particularly preferably between 150 and 50,000 FCC units.

The activity of maltase is defined in units, wherein one unit is the amount of maltase which will convert maltose to D-glucose at a rate of one milligram per minute at 37° C. and a pH of 4.0 in a 10% maltose solution by weight.

Where the agent according to the present invention also contains maltase, the activity per dose unit should be between 100 and 100,000 units, preferably between 200 and 50,000 units and particularly preferably between 500 and 20,000 units

Also for the other enzymes mentioned, the standard test conditions and the way in which the enzyme activities are to be determined are known and can be read up by specialists in the field.

Insofar as one or more of the optional enzymes are added to the agent according to the present invention, they—as is the case for the 5-D-fructose dehydrogenase and the glucose isomerase—should be used in sufficient amounts so that they can develop a sufficient enzyme activity for the intended purpose, e.g. sufficient invertase, so that an amount of sucrose usually ingested with a normal meal (e.g. 15 g) can be cleaved, and/or lactase, so that an amount of lactose usually ingested with a normal meal (e.g. 10 g) can be cleaved.

The enzymes used can be for example in solid form, e.g. as crystalline or amorphous granules or powders, as a paste or as a liquid, as well as in other forms. In some embodiments, the enzyme is a free enzyme. In other embodiments, the enzyme may e.g. be immobilized on substrate, which can be powderized if necessary before the enzyme is used in accordance with the invention.

If the agent according to the present invention is added to a foodstuff before consumption or during production, the activity of 5-D-fructose dehydrogenase should be between 10 and 250,000 units, preferably between 25 and 150,000 units and particularly preferably between 50 and 100,000 units per gram of fructose and glucose combined contained in the foodstuff and the activity of the glucose isomerase should be between 0.01 and 20,000 GIU, preferably between 0.05 and 10,000 GIU and particularly preferably between 0.1 and 1000 GIU per gram of glucose in the foodstuff.

It may be advantageous to add an electron acceptor to the agent according to the present invention at e.g. a ratio (acceptor:substrate) of 1:1 to 1:1,000, preferably at a ratio of 1:2 to 1:200, particularly preferably at a ratio of 1:10 to 1:50. Examples of suitable acceptors which may be used include NAD+, NADP+, FAD+, vitamins, such as vitamin C, vitamin E or vitamin A, ferricyanide, ketones, aldehydes, 2,6-dichlorophenolindophenol, phenazine methosulfate, nitroblue tetrazolium (including mixtures thereof), but are not limited thereto.

The physiologically present electrolytes should be sufficient for the function of glucose isomerase. But it may also be advantageous to add electrolytes to the agent according to the present invention, e.g. in an amount of 0.0001% to 0.1% of the substrate (glucose). Examples of electrolytes include, but are not limited to, MgSO₄, Na₂CO₃, NaHCO₃, NaOH, Na₂SO₄, MgCO₃, H₂SO₄, NaS₂O₃, NaS₂O₅ (including mixtures thereof).

It may also be advantageous to add metal ions, especially cations, such as Mn²⁺, Mg²⁺, Ca²⁺, Zn²⁺, Fe²⁺, Co²⁺ or Cu²⁺, including mixtures thereof, to the agent according to the present invention, namely preferably in a molar ratio of 10⁻⁶ to 10⁻². For the above mentioned (xylose) glucose isomerase which is described by Yamanaka, especially Mn²⁺ is a suitable cation.

Capsule sizes mentioned below refer to the size definitions used by Capsugel Belgium BVBA, Bornem, Belgium. The size of the capsules should be chosen according to the specific formulation of the agent.

A composition according to the present invention for the production of capsules (e.g. of size 3) may consist of 55 mg of 5-D-fructose dehydrogenase with an activity of 1000 units/mg, 50 mg of glucose isomerase with an activity 1 GIU/mg and 55 mg of dicalcium phosphate per capsule.

A further example for a dosage form according to the present invention consists of capsules (size 00) that contain 165 mg of 5-D-Fructose dehydrogenase with an activity of 1000 units/mg, 150 mg of glucose isomerase with an activity of 1 GIU/mg and 155 mg of dicalcium phosphate per capsule.

In a further composition example, a capsule of size 0 may contain 250 mg of 5-D-fructose dehydrogenase with an activity of 90 units/mg and 20 mg of glucose isomerase with an activity of 1 GIU/mg and 50 mg of dicalcium phosphate.

A further example for the production of capsules of size 00 may contain 370 mg of 5-D-fructose dehydrogenase with an activity of 90 units/mg, 30 mg of glucose isomerase with an activity of 1 GIU/mg and 70 mg of dicalcium phosphate.

Another example for the dosage form according to the present invention consists of capsules of size 00 which contain 110 mg of 5-D-fructose dehydrogenase with an activity of 500 units/mg, 50 mg of glucose isomerase with an activity of 1 GIU/mg, 100 mg of invertase with an activity of 200 SU units/mg, 90 mg of lactase with an activity of 100 FCC units/mg and 120 mg of dicalcium phosphate.

The invention may for example contain between 10 and 5 million units of 5-D-fructose dehydrogenase and between 0.01 and 100,000 GIU (=glucose isomerase units) of glucose isomerase per dose unit. In addition, suitable additives may be used in the required amount.

The invention may be provided for medical purposes and non-medical purposes, e.g. as a pharmaceutical composition, medical device, foodstuff or special food-stuff.

With the agent according to the present invention, afflictions and impairments of health that are caused by diabetes in its various degrees of severity can be significantly reduced or eliminated. The invention disclosed herein is especially suitable for the therapeutic treatment of diabetes. It is also suitable for use in methods for the therapeutic treatment of the human or animal body in which an uncontrolled increase in the level of blood sugar is to be prevented or a decrease in the level of blood sugar is intended.

In the following claims, the term “glucose equivalent-containing” refers to all substances and foodstuffs that contain glucose (a) as glucose per se, (b) in a form from which glucose can be released in the digestive tract (e.g. by cleavage as glucose from a saccharide chain containing at least two saccharide monomers, such as sucrose), (c) in a form that can be converted to glucose, e.g. as fructose per se, or (d) in a form that can be released in the digestive tract and converted to glucose, e.g. as a saccharide chain containing at least two saccharide monomers, at least one of which can be cleaved from the saccharide chain as fructose.

In the following claims, the term “total glucose” refers to the total content of glucose in a foodstuff (a) as glucose per se, (b) in a form from which glucose can be released in the digestive tract (e.g. by cleavage as glucose from a saccharide chain containing at least two saccharide monomers, such as sucrose), (c) in a form that can be converted to glucose, e.g. as fructose per se, or (d) in a form that can be released in the digestive tract and converted to glucose, e.g. a saccharide chain containing at least two saccharide monomers, at least one of which can be cleaved from the saccharide chain as fructose.

In the following claims, the term “effective glucose content” of an item refers to the effective amount of total glucose in that item, taking into account the prior action of glucose and fructose converting enzymes that have been added and the future action of glucose and fructose converting enzymes that have been added to the item. Thus, for example, a foodstuff having a given glucose content and having microencapsulated glucose isomerase and microencapsulated 5-D-fructose dehydrogenase incorporated therein will have a lower effective glucose content than a foodstuff which lacks the microencapsulated glucose isomerase and 5-D-fructose dehydrogenase but is otherwise identical, since release of the glucose isomerase and 5-D-fructose dehydrogenase after ingestion will result in at least a portion of the glucose in the foodstuff being converted to 5-keto D-fructose.

In the following claims, the term “total fructose” refers to the total content of fructose in a foodstuff (a) as fructose per se, (b) in a form from which fructose can be released in the digestive tract (e.g. by cleavage as fructose from a saccharide chain containing at least two saccharide monomers), (c) in a form that can be converted to fructose, e.g. as glucose per se, or (d) in a form that can be released in the digestive tract and converted to fructose, e.g. a saccharide chain containing at least two saccharide monomers, at least one of which can be cleaved from the saccharide chain as glucose.

1-230. (canceled)

231. A method of treating diabetes, reducing the effect of diabetes on a mammalian body, reducing the effect of D-glucose in a mammalian body, or reducing the effect of total glucose in a mammalian body, comprising administering to a mammalian subject in need of such treatment or reduction an efficacious amount of a combination of 5-D-fructose dehydrogenase and a glucose isomerase.

232. A method according to claim 231, further comprising administering to said subject a third enzyme selected from the group consisting of invertase, maltase, lactase, alpha-amylase, beta-amylase, glucoamylase, pullulanase, isoamylase, amyloglucosidase, cyclomaltodextrin glucanotransferase and combinations thereof.

233. A method according to claim 231, wherein said 5-D-fructose dehydrogenase is administered prior to eating, immediately prior to eating, concurrently with eating, immediately after eating or after eating.

234. A method according to claim 233, wherein said glucose isomerase is administered prior to eating, immediately prior to eating, concurrently with eating, immediately after eating or after eating.

235. A method according to claim 231, wherein said administration is oral administration.

236. A method according to claim 231 wherein said method is part of a program of therapeutic treatment or management of diabetes.

237. A method according to claim 231, wherein said effect of D-glucose or total glucose is selected from the group consisting of a deleterious effect on the health of said mammalian subject, hyperglycemia, and a complication of diabetes.

238. A method according to claim 237, wherein said complication of diabetes is selected from the group consisting of diabetic angiopathy, macroangiopathy, coronary heart disease, microangiopathy, retinopathia diabetica, maculopathy, nephropathia diabetica, gangrene, Gastroparesis diabeticorum, impaired function of the small intestine, constipation, stool incontinence, diabetic neuropathy, diabetic autonome neuropathy and diabetic foot.

239. A method according to claim 231, wherein said diabetes is selected from the group consisting of diabetes type I, LADA diabetes (latent autoimmune diabetes in adults), diabetes type II, pregnancy-induced diabetes, impaired glucose tolerance, and a minor disorder of blood sugar metabolism.

240. A kit comprising 5-D-fructose dehydrogenase and instructions explaining how to use said 5-D-fructose dehydrogenase in combination with a glucose isomerase to treat diabetes, to reduce the effects of diabetes on a mammalian body, to reduce the effects of glucose in a mammalian body or to reduce the effects of total glucose in a mammalian body.

241. A kit according to claim 240 wherein said instructions further explain how to use a third enzyme selected from the group consisting of invertase, maltase, lactase, alpha-amylase, beta-amylase, glucoamylase, pullulanase, isoamylase, amyloglucosidase, cyclomaltodextrin glucanotransferase and combinations thereof in combination with said 5-D-fructose dehydrogenase and said glucose isomerase to treat diabetes, to reduce the effects of diabetes on a mammalian body, to reduce the effects of glucose in a mammalian body or to reduce the effects of total glucose in a mammalian body.

242. A kit according to claim 240 which further comprises a second enzyme selected from the group consisting of a glucose isomerase, invertase, maltase, lactase, alpha-amylase, beta-amylase, glucoamylase, pullulanase, isoamylase, amyloglucosidase, cyclomaltodextrin glucanotransferase and mixtures thereof.

243. A method for preparing a reduced total glucose foodstuff, comprising contacting a glucose-containing foodstuff or foodstuff precursor with 5-D-fructose dehydrogenase, a glucose isomerase and at least one enzyme that cleaves glucose or fructose from saccharide chains having at least two saccharide monomers, and completing any additional steps necessary to prepare the foodstuff, provided that said foodstuff precursor is not a dough and said foodstuff is not a dough or a baked foodstuff.

244. A process for preparing a 5-D-fructose dehydrogenase- and glucose isomerase-containing foodstuff, the process comprising adding to a foodstuff or foodstuff precursor 5-D-fructose dehydrogenase and a glucose isomerase, at least some of said 5-D-fructose dehydrogenase being in a form in which said 5-D-fructose dehydrogenase will be released in active form to act on fructose which is present in or released from said foodstuff after said foodstuff s ingested by a subject, and at least one of the following being true:
(a) at least some of said glucose isomerase is in an active form in which said glucose isomerase can act on a substrate which is
(i) present in or released from said foodstuff precursor or
(ii) present in or released from said foodstuff before ingestion of said foodstuff, and
(b) at least some of said glucose isomerase is present in said foodstuff in a form in which said glucose isomerase will be released in active form to act on a substrate which is present in or released from said foodstuff after ingestion of said foodstuff,
and completing any additional steps necessary to prepare said foodstuff, whereby to obtain said 5-D-fructose dehydrogenase- and glucose isomerase-containing foodstuff.

245. A process according to claim 244, wherein said 5-D-fructose dehydrogenase- and glucose isomerase-containing foodstuff further comprises at least one enzyme selected from the group consisting of invertase, maltase, lactase, alpha-amylase, beta-amylase, glucoamylase, pullulanase, isoamylase, amyloglucosidase, cyclomaltodextrin glucanotransferase and mixtures thereof.

246. A method according to claim 231, wherein at least one of said 5-D-fructose dehydrogenase and said glucose isomerase is isolated from a natural, synthetic, or recombinant source.

247. A process according to claim 244, wherein at least one of said 5-D-fructose dehydrogenase and said glucose isomerase is isolated from a natural, synthetic, or recombinant source.

248. A kit according to claim 242, wherein said second enzyme is a glucose isomerase, and at least one of said 5-D-fructose dehydrogenase and said glucose isomerase is isolated from a natural, synthetic, or recombinant source.

249. A method according to claim 243, wherein at least one of said 5-D-fructose dehydrogenase and said glucose isomerase is isolated from a natural, synthetic, or recombinant source.
